(19) United States
(12) Patent Application Publication
(10) Pub. No.: US 2014/0206717 A1
(43) Pub. Date: Jul. 24, 2014

(54) USE OF INORGANIC MATRIX AND ORGANIC POLYMER COMBINATIONS FOR PREPARING STABLE AMORPHOUS DISPERSIONS

(71) Applicants: John Higgins, Malvern, PA (US); David C. Dubost, Collegeville, PA (US)

(72) Inventors: John Higgins, Malvern, PA (US); David C. Dubost, Collegeville, PA (US)

(21) Appl. No.: 14/238,874

(22) PCT Filed: Aug. 10, 2012
(86) PCT No.: PCT/US12/50221

§ 371 Date: Feb. 14, 2014

Related U.S. Application Data
(60) Provisional application No. 61/524,033, filed on Aug. 16, 2011.

(51) Int. Cl. A61K 47/02; A61K 47/38; A61K 31/437; A61K 9/10; A61K 31/405; A61K 31/277

(52) U.S. Cl. CPC A61K 47/02; A61K 31/405; A61K 31/277; A61K 31/437; A61K 9/10; A61K 47/38
USPC 514/303; 514/420; 514/521

(57) ABSTRACT

The present invention relates to methods for processing pharmaceutically active substances having poor water solubility in the presence of an inorganic matrix, e.g., magnesium aluminometasilicate, and a secondary polymer as a means of converting the crystalline API to substantially amorphous and stable form, i.e., the crystallinity is less than 5%. The methods of the invention result in more complete amorphization, increased solubility, drug loading and stability as compared to typical amorphization or literature methods.

### CROSS-REFERENCE TO RELATED APPLICATIONS

Not Applicable

### FIELD OF THE INVENTION

The present invention relates to methods for preparing highly stable amorphous dispersions of poorly soluble active pharmaceutical ingredients (APIs) via processing with an inorganic matrix, e.g., magnesium aluminometasilicate, and a secondary polymer, and compositions made thereby. The methods of the invention result in more complete amorphization, increased solubility, drug loading and stability as compared to processing with an inorganic matrix alone.

### BACKGROUND OF THE INVENTION

Poor aqueous solubility can be a serious problem for achieving adequate drug bioavailability. In particular, poor solubility often limits oral absorption from the GI tract. Drug solid state forms with optimal solubility/dissolution rates can result in better absorption from the GI tract. It follows that using a drug form with optimal solubility also can allow for similar plasma levels as seen with a larger dose of a less soluble form. Therefore, enhancing the dissolution, solubility and bioavailability of poorly soluble drugs is of great interest in the art.

In general, amorphous forms of a substance show a higher solubility and/or dissolution rate than crystalline forms of the same substance. The higher dissolution rate/solubility of amorphous phases as well as the potentially obtained oversaturated solution can result in better bioavailability as compared to an associated crystalline form. More soluble amorphous phases are desirable for both human solid dosage forms and for use in formulations (suspensions) for preclinical toxicology studies, where large exposure margins often are required.

Frequently, amorphous drugs will convert to the lower energy crystalline phase, resulting in a drop in solubility. See Hancock and Zografi, 1997, J. Pharm Sci. 86:1-12. It is well known that crystallization can be suppressed by dissolving the drug into an amorphous polymer, thus forming a stablized “amorphous solid dispersion”. Drug-polymer solid dispersions can be prepared via several means, including melt extrusion and spray drying.

Many other approaches have been taken to achieve a desired level of drug solubility and dissolution rate. These approaches have been based on preparations with increased surface area (micronised powders), molecular inclusion complexes (cyclodextrines and derivatives), co-precipitates with water-soluble polymers (PEG, polozamers, PVP, HPMC) and non-electrolytes (urea, mannitol, sugars etc.), micellar solutions in surfactant systems (Cremophor™, Tween™, Gellucires™), and multilayer vesicles (liposomes and niosomes). Dispersed colloidal vehicles, such as oil-in-water, water-in-oil and multiple (O/W/O or W/O/W) emulsions, microemulsions and self-emulsifying compositions also have been used to improve bioavailability of poorly soluble molecules. Reducing the particle size of a substance also can be useful for increasing the dissolution rate of an active pharmaceutical ingredient (API), as a reduction in particle size correlates to an increase in surface area. In particular, reducing the particle size reduction to the nanometer size range is highly desirable.

Another method for preparing an amorphous solid dispersion has been reported, where the drug was processed by milling along with magnesium aluminometasilicate to generate the amorphous drug phase (See Gupta, 2003, J. Pharm. Sci. 92:536-551). However, it has now been shown that complete amorphization is not always initially attained or in some cases, drug crystallizes from the matrix in a short time. This suggests that the in vivo performance would not be optimal. These observations render this approach inappropriate for use in pre-clinical or clinical formulations. Means for generating more stable compositions which could circumvent these observations would be of great utility.

Citation or identification of any reference in this section or any other section of this application shall not be construed as an indication that such reference is available as prior art to the present invention.

### SUMMARY OF THE INVENTION

The present invention relates to methods for the preparation of stable amorphous dispersions of pharmaceutically active substances with improved aqueous solubility via processing in the presence of an inorganic matrix, e.g., magnesium aluminometasilicate, and a secondary polymer, and compositions made thereby. The key element of this invention (addition of a secondary polymer) results in more complete amorphization, better physical stability and increased solubility/dissolution as compared to reported literature methods using inorganic matrices alone.

In accordance with the present invention, a method is disclosed for producing a substantially amorphous stable drug product comprising preparing an amorphous dispersion, e.g., by milling, an active pharmaceutical ingredient (API) in the presence of an inorganic matrix, e.g., magnesium aluminometasilicate, and a secondary polymer. A composition is obtained in which the drug product has a purity by chromatographic analysis (chemical purity) of at least 95%, 98% or 99%, and the drug product is substantially free of any crystalline material, i.e., contains less than about 5%, or 2% or 1% crystalline material. The methods of invention are suitable for any method for preparing an amorphous dispersion of API, including, but not limited to, spray drying, extrusion, or milling

In certain embodiments, the inorganic matrix is a silicate, a calcium phosphate, or an inorganic clay (e.g., kaolin). In one aspect, the inorganic matrix is magnesium aluminosilicate such as magnesium aluminometasilicate. In certain embodiments, the secondary polymer is a cellulose, acrylate, poloxamer, vinyl homopolymer or copolymer, polyethylene glycol, aminosaccharide or polyethylene oxide. Examples of cellulose include, but are not limited to, ethyl(hydroxyethyl)cellulose, hydroxypropyl methylcellulose, hydroxyethyl cellulose. The cellulose can be modified with one or more hydrophobic/hydrophilic groups (e.g., a carboxylic acid) or a methacrylic acid copolymer. Examples of acrylate include, but are not limited to, methacrylic acid copolymer. In one aspect, the secondary polymer is hydroxypropyl methylcellulose functionalized with a carboxylic acid (e.g., hydroxypropyl methylcellulose acetate succinate or hydroxypropyl methylcellulose phthalate).

Examples of drug product/API include, but are not limited to, megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline. In certain aspects, the drug product is megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, or budesnonide. In other aspects, the drug product is 5″-chloro-N-[(5,6-dimethoxypyridin-2-yl)methyl]-2,2′:5′,3″-terpyridine-3′-carboxamide, N¹-(1-cyanocyclopropyl)-4-fluoro-N²-{(1S)-2,2,2-trifluoro-1-[4′-methylsulfonyl]-1,1′-biphenyl-4-yl}ethyl}-L-leucinamide, or 3-Chloro-5-{[5-chloro-1-(1H-pyrazolo[3,4-b]pyridin-3-ylmethyl)-1H-indazol-4-yl]oxy}benzonitrile.

The present invention is also directed to amorphous drug product produced by the methods of the invention. In certain embodiments, the amorphous drug product contains substantially no crystalline content (e.g., less than 5%, 2% or 1%).

The present invention is also directed to amorphous drug product comprising API, an inorganic matrix and a secondary polymer. The API, inorganic matrix and secondary polymer are as defined in the embodiments of the methods described above. In certain embodiments, the amorphous drug product contains substantially no crystalline content (e.g., less than 5%, 2% or 1%).

The present invention also relates to a formulation containing the amorphous drug product in the form of a liquid suspension or solid dosage form.

### DETAILED DESCRIPTION OF THE INVENTION

The present invention relates to methods for the processing of active pharmaceutical ingredient (API), for example by milling, in the presence of an inorganic matrix, e.g., magnesium aluminometasilicate, and a secondary polymer, final amorphous drug product obtained using the methods of the invention, and formulations containing the amorphous drug product. The methods of the invention result in more complete amorphization, enhanced solubility and greater physical stability as compared to other methods using the synthetic magnesium aluminometasilicate, Neusilin®, reported in literature. As demonstrated in the Examples, amorphous Indomethacin—Neusilin® dispersions made in the absence of a secondary polymer rapidly crystallize when dispersed into simulated intestinal fluid. As compared to classical amorphization processes such as spray drying, the present invention results in high efficiency and avoidance of solvents.

According to the present invention, substantially amorphous drug product is obtained by processing crystalline API together with an inorganic matrix and a secondary polymer until the mixture is substantially free of any crystalline material. The resulting drug product also is highly pure via chromatographic analysis (>95% pure active).

As used herein, the term “amorphous” means a solid body devoid of long-range crystalline order. Such a lack of crystalline order can be detected and monitored. e.g., by X-ray diffraction (XRD), FT-Raman spectroscopy, and differential scanning calorimetry (DSC).

As used herein, the phrase “substantially amorphous form” means the form contained in the amorphous solid solution is in the amorphous state, e.g., there is a minimum of 95% of active ingredient in the amorphous state in the amorphous solid solution, preferably 98% and more preferably 99% of the active ingredient, or even 100% in the amorphous state. The phrase “amorphous active ingredient” is also intended to mean a non-crystalline active pharmaceutical ingredient.

As used herein, the term “milling” means grinding between two surfaces. Milling can be conducted with a mortar and pestle or a milling process such as ball milling, roller milling, or gravatory milling.

As used herein, the phrase “poorly soluble active agents” means active agents having a solubility in at least one liquid dispersion medium of less than about 30 mg/ml, preferably less than about 20 mg/ml, preferably less than about 10 mg/ml, preferably less than about 1 mg/ml, or preferably less than about 0.1 mg/ml. Such active agents tend to be eliminated from the gastrointestinal tract before being absorbed into the circulation. Moreover, poorly water soluble active agents tend to be unsafe for intravenous administration techniques, which are used primarily in conjunction with highly water soluble active agents.

As used herein, the terms “preparing an amorphous dispersion” and “processing” mean utilizing any method suitable for preparing amorphous drug product, including, but not limited to, extrusion, spray drying and milling.

### Inorganic Matrix

An inorganic matrix useful in the methods of the invention generally possesses a large surface area and is of a porous nature and is generally amorphous in and of itself. The amorphous inorganic matrix acts in an analogous way as a typical organic polymer has the ability to absorb active pharmaceutical ingredient. In certain embodiments, the inorganic matrix is a silicate (e.g., calcium silicate, magnesium silicate, magnesium trisilicate), a calcium phosphate (e.g., di- or tri-basic calcium phosphate), or an inorganic clay (e.g., kaolin). In one aspect, the inorganic matrix is magnesium aluminosilicate such as magnesium aluminometasilicate.

In one aspect, the inorganic matrix is magnesium aluminometasilicate amorphous. Magnesium aluminometasilicate may be represented by the general formula Al₂O₃.MgO.xSiO₂ nH₂O, wherein x is in a range of about 1.5 to about 2, and n satisfies the relationship 0≦n≦10. In certain embodiments, the magnesium aluminometasilicate amorphous is synthetic. In one embodiment, the magnesium aluminometasilicate amorphous is a synthetic version sold by Fuji Chemical Industry Co. Ltd. under the brand name Neusilin®.

Other examples of inorganic matrices suitable for use in the present invention include, but are not limited to, anhydrous silicic acid, calcium carbonate, calcium sulphate, magnesium carbonate, magnesium oxide and co-processed insoluble excipients. Silicon dioxide-colloidal (e.g., Syloid® 244, W.R. Grace & Co., Columbia, Md.; Sipernat®, Evonik Degussa Corporation, Parsipanny, N.J.) or fumed (prepared by hydrolysis of silicone alides—Cab-O-Sil M5®, Cabot Corporation, Boston, Mass., or Aerosil® 200/300, Evonik Degussa Corporation, Parsipanny, N.J.), zeolites, talcite, bentonite, etc.

### Secondary Polymers

The addition of the secondary polymer serves to aid in amorphization and increase solubility. Without being bound by any mechanism, the increased solubility may be due in part to suppression of seed crystal formation which would lead to crystallization. Secondary polymers useful in the methods of the invention include, but are not limited to cellulosic polymers and vinyl homopolymers and copolymers.

In certain embodiments, the secondary polymer is a cellulose, acrylate, poloxamer, vinyl homopolymer or copolymer, polyethylene glycol, aminosaccharide or polyethylene oxide.

Examples of cellulose (cellulosic polymers), which can be modified with one or more hydrophobic/hydrophilic groups (e.g., a carboxylic acid) or a methacrylic acid copolymer, include, but are not limited to alkylcelluloses, e.g., methylcellulose; hydroxyalkylcelluloses, e.g., hydroxymethylcellulose, hydroxyethylcellulose (Natrosol™, Ashland, Covington, Ky.), hydroxypropylcellulose, hydroxybutylcellulose and weakly substituted hydroxypropylcellulose; hydroxyalkylalkylcelluloses, e.g., ethyl(hydroxyethyl)cellulose, hydroxyethylmethylcellulose and hydroxypropylmethylcellulose (e.g., Methocel™, types A, E, K, F, Dow Wolff Cellulosics GmbH, Bomlitz, Germany); carboxyalkylcelluloses, e.g., carboxymethylcellulose; carboxyalkylcellulose salts, e.g., sodium carboxymethylcellulose; carboxyalkylalkylcelluloses, e.g., carboxymethylethylcellulose; esters of cellulose derivatives, e.g., hydroxypropylmethylcellulose phthalate, hydroxypropylmethylcellulose acetate succinate (e.g., AQOAT® (Shin-Etsu, Tokyo, Japan)), and cellulose acetate phthalate-hydroxypropylcellulose (e.g., KLUCEL® (Ashland, Covington, Ky.)).

In one aspect, the secondary polymer is hydroxypropyl methylcellulose functionalized with a carboxylic acid (e.g., hydroxypropyl methylcellulose sucinate or hydroxypropyl methylcellulose phthalate).

Examples of acrylate include polyacrylates including, but are not limited to, methacrylic acid copolymer, polymethacrylates (Eudragit® L-100-55 and Eudragit® E-100, Evonik Degussa Corporation, Parsipanny, N.J.), polyacrylic acid (Carbopol®, The Lubrizol Corporation, Wickliffe, Ohio).

Examples of vinyl homopolymers and copolymers include, but are not limited to, polymers of N-vinylpyrrolidone, in particular povidone, copovidone, polyvinyl alcohol, and polyvinylpyrrolidone (Kollidon™, PVP and PVP-VA, BASF SE, Ludwigshafen, Germany).

Examples of other types of synthetic polymers include, but are not limited to, polyethylene oxide (Polyox™, Dow Chemical Company, Midland, Mich.), polyethyleneglycols of various molecular weights, polyethylene-/polypropylene-/polyethylene-oxide block copolymers and natural gums and polysaccharides—Xanthan gum (Keltrol™, CP Kelco, Atlanta, Ga.), carrageenan, locust bean gum, acacia gum, chitosan, alginic acid, hyaluronic acid, pectin, etc. Suitable polyethyleneglycols are especially Polyethyleneglycol 8000 and Polyethyleneglycol 6000. A suitable polyethylene-/polypropylene-/polyethylene-oxide block copolymer is in particular Pluronic F68.

Inorganic Matrix/Secondary Polymer Combination

The inorganic matrix/secondary polymer combination can be from about 25% to about 99% by weight of the total load, more preferably about 50% to about 90% or about 60% to about 80%.

The ratio of inorganic matrix to secondary polymer can be from 20:1 to 1:1, 10:1 to 1:1, 5:1 to 1:1, 1:1 to 1:5, 1:1 to 1:10, or 1:1 to 1:20 by weight.

### Drugs/API

Active pharmaceutical ingredients used in the methods of the present invention include all those compounds known to have an effect on humans or animals that also have low water solubility, e.g., less than 50 μg/ml,. Such compounds include all those that can be categorized as Class 2 under the Biopharmaceutical Classification System (BCS) set out by the United States Food and Drug Administration (FDA).

Examples of APIs suitable for use with the methods of the invention include, but are not limited to, megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Preferably, the API is megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, and verapamil. More preferably, such compounds include megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, or budesnonide.

In one aspect, the API is indomethacin or itraconizole. In another aspect, the API is 5″-chloro-N-[(5,6-dimethoxypyridin-2-yl)methyl]-2,2′:5′,3″-terpyridine-3′-carboxamide (U.S. Patent Application Publication No. 20100035931)

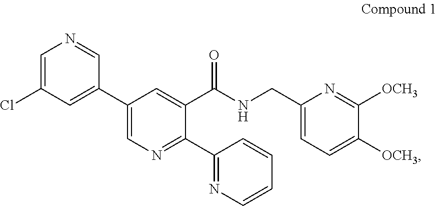 [SMILES: COc1ccc(CNC(=O)c2cc(-c3cncc(Cl)c3)cnc2-c2ccccn2)nc1OC]

N¹-(1-cyanocyclopropyl)-4-fluoro-N²-{(1S)-2,2,2-trifluoro-1-[4′-methylsulfonyl]-1,1′-biphenyl-4-yl}ethyl}-L-leucinamide (U.S. Patent Application Publication No. 20030232863)

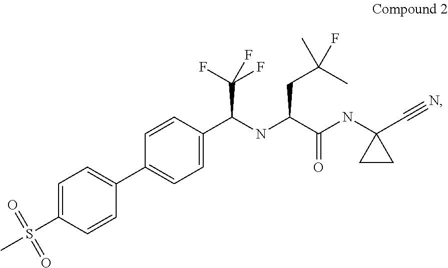 [SMILES: CC(C)(F)C[C@H](N[C@@H](c1ccc(-c2ccc(S(C)(=O)=O)cc2)cc1)C(F)(F)F)C(=O)NC1(C#N)CC1]

or 3-Chloro-5-{[5-chloro-1-(1H-pyrazolo[3,4-b]pyridin-3-ylmethyl)-1H-indazol-4-yl]oxy}benzonitrile (U.S. Pat. No. 7,781,454)

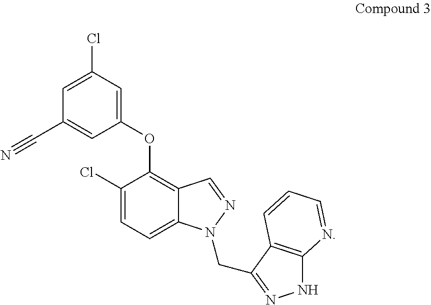 [SMILES: N#Cc1cc(Cl)cc(Oc2c(Cl)ccc3c2cnn3Cc2n[nH]c3ncccc23)c1]

The API is present in a range from about 1% to about 75% by weight, and more preferably the API is present in a range from about 10% to about 50% by weight, or 20% to about 40%.

### Processing

Although the compositions described herein may be prepared by any process for amorphization including spray drying or extrusion, milling processes are preferred due to the solvent-free process and low temperatures employed.

Milling

Milling is a pharmaceutical unit operation designed to break a solid material (i.e., an API) into smaller particles. The smaller particles are often also of more uniform size distribution. In the methods of the invention, amorphous API can be prepared by milling or micronization until the crystalline API is converted to amorphous material, as can be determined by XRD, FT-Raman spectroscopy or DSC. Any milling process can be used in the methods of the invention. Milling techniques for pharmaceuticals are described in Remington's Pharmaceutical Sciences, 20^{th} edition, edited by A. R. Gennaro, Mack Publishing Co., 2000. The milling process can be a dry milling or a wet milling process. However, dry milling is preferred. Such milling has been traditionally carried out in pharmacy practice by compounding using a pestle and mortar. The milling procedure may be carried out by milling machines known in the art. Suitable milling machines include various types of ball mills (preferred), roller mills, cryo mills, gyratory mills, and the like. Alternatively, the milling may be carried out using commercially available milling machines, such as jet mill or rotor stator colloid mills, which grind drugs into powders that have particle sizes ranging from 0.1 μm to 25 μm. Wet media mills, such as described in U.S. Pat. Nos. 5,797,550 and 4,848,676, are generally used to mill or grind relatively large quantities of materials.

One example of a commercially available milling machine suitable for carrying out the process of the present invention is the Retsch mill (Retsch GMBH, Germany), which is a common oscillating ball mill. This type of mill provides sufficient energy and residence time such that a typical crystalline API/Neusilin®/secondary polymer mixture can be converted to a pure amorphous phase in a reasonable time frame.

The period of milling using the Retsch mill will vary depending on the size of the mill, the speed of rotation of the main shaft, the type of feed material, and the quantity of feed material. The effects of these variables are well known in the art and the invention may be worked over a range of these variables. Typically, the period of milling ranges from about 15 minutes to 300 minutes or up to 10 hours.

The solid solution thus obtained by one of the processes according to the invention can be milled so as to obtain a fine powder (particle size <300 μm).

Spray Drying

Spray drying and spray coating broadly refer to processes involving breaking up liquid mixtures into small droplets (atomization) and rapidly removing solvent from the mixtures in a vessel such as a spray-drying apparatus or a fluidized bed- or pan-coater where there is a strong driving force for evaporation of solvent from the droplets. In the case of spray-coating the droplets impinge on a particle, bead, pill, tablet, or capsule, resulting in a coating comprising the solid amorphous dispersion. Spray-coating may also be conducted on a metal, glass or plastic surface and the coated layer may subsequently be removed and milled to the desired particle size. In the case of spray-drying, the droplets generally dry prior to impinging on a surface, thus forming particles of solid amorphous dispersion on the order of 1 to 100 micrometers in diameter. The strong driving force for solvent evaporation is generally provided by maintaining the partial pressure of solvent in the spray-drying apparatus well below the vapor pressure of the solvent at the temperature of the drying droplets. This is accomplished by either (1) maintaining the pressure in the spray-drying apparatus at a partial vacuum (e.g., 0.01 to 0.50 atm); (2) mixing the liquid droplets with a warm drying gas; or (3) both (1) and (2).

Suitable spray-drying techniques are described, for example, by K. Masters in “Spray Drying Handbook”, John Wiley & Sons, New York, 1984 and Remington's Pharmaceutical Sciences, 20^{th} edition, edited by A. R. Gennaro, Mack Publishing Co., 2000. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of suitable spray drier using rotary atomization is the Mobile Minor spray drier, manufactured by Niro, Denmark. The hot gas can be, for example, air, nitrogen or argon.

Generally, the temperature and flow rate of the drying gas is chosen so that polymer/drug solution droplets are dry enough by the time they reach the wall of the apparatus that they are essentially solid, so that they form a fine powder and do not stick to the apparatus wall. The actual length of time to achieve this level of dryness depends on the size of the droplets. Droplet sizes generally are larger than about 1 μm in diameter, with 5 to 100 μm being typical. The large surface-to-volume ratio for the droplets and the large driving force for evaporation of solvent leads to actual drying times of a few seconds or less. For some mixtures of drug/polymer/solvent this rapid drying is critical to the formation of a relatively uniform, homogeneous composition as opposed to an undesirably separation into drug-rich and polymer-rich phases. Such dispersions having a homogenous composition can be considered solid solutions and may be supersaturated in drug.

Solidification times should be less than 100 seconds, preferably less than a few seconds, and more preferably less than 1 second. In general, to achieve such rapid solidification of the drug/polymer solution, it is preferred that the diameter of droplets formed during the spray-drying process are less then 100 μm, preferably less than 50 μm, and most preferably less than 25 μm. The so-formed solid particles resulting from solidification of these droplets generally tend to be 2 to 40 μm in diameter.

Following solidification, the solid powder typically remains in the spray-drying chamber for 5 to 60 seconds, evaporating more solvent. The final solvent content of the solid dispersion as it exits the dryer should be low, since low solvent content tends to reduce the mobility of drug molecules in the dispersion, thereby improving its stability. Generally, the residual solvent content of the dispersion should be less than 10 wt % and preferably less than 2 wt %.

Solvents suitable for spray-drying may be essentially any organic compound or mixtures of an organic compound and water in which the drug and polymer are mutually soluble. Because the invention utilizes low water solubility drugs, water alone is generally not a suitable solvent. However, mixtures of water and organic compounds are often suitable. Preferably, the solvent is also relatively volatile with a boiling point of 150° C. or less. However, in those cases where the solubility of the drug in the volatile solvent is low, it may be desirable to include a small amount, say 2 to 25 wt %, of a low volatility solvent such as N-methylpyrrolidone (NMP), dimethylsulfoxide (DMSO) or dimethylacetamide (DMAc) in order to enhance drug solubility. Preferred solvents include alcohols such as methanol, ethanol, n-propanol, isopropanol, and butanol; ketones such as acetone, methyl ethyl ketone and methyl isobutyl ketone; esters such as ethyl acetate and propylacetate; and various other solvents such as acetonitrile, methylene chloride, toluene, and 1,1,1-trichloroethane.

Preferably, the particles of the invention are obtained by spray drying using an inlet temperature between about 100° C. and about 400° C. and an outlet temperature between about 50° C. and about 130° C.

Extrusion

Extrusion refers to processes whereby drug product is forced through pharmaceutical extruders. See, e.g., Repka, Amer. Pharm. Rev., Sep. 2009, 18-26. A melt-extrusion process comprises heating a mixture of drug and polymers until a homogenous melt is obtained, forcing the melt through one or more nozzles; and cooling the melt till it solidifies.

The terms “melt” and “melting” should be interpreted broadly. These terms not only mean the alteration from a solid state to a liquid state, but can also refer to a transition to a glassy state or a rubbery state, and in which it is possible for one component of the mixture to get embedded more or less homogeneously into the other. In particular cases, one component will melt and the other component(s) will dissolve in the melt thus forming a solution, which upon cooling may form a solid solution having advantageous dissolution properties.

One of the most important parameters of melt extrusion is the temperature at which the melt-extruder is operating. Operating temperatures can range between about 120° C. and about 300° C.

The throughput rate is also of importance because even at relatively low temperatures the water-soluble polymer may start to decompose when it remains too long in contact with the heating element.

It will be appreciated that the person skilled in the art will be able to optimize the parameters of the melt extrusion process within the above given ranges. The working temperatures will also be determined by the kind of extruder or the kind of configuration within the extruder that is used. Most of the energy needed to melt, mix and dissolve the components in the extruder can be provided by the heating elements. However, the friction of the material within the extruder may also provide a substantial amount of energy to the mixture and aid in the formation of a homogenous melt of the components.

### Formulation

The inorganic matrix/drug/secondary polymer dispersions can be formulated into any type of liquid or solid or semi-solid dosage form for administration by means such as oral and subcutaneous routes. Liquid preparations suitable for oral administration (e.g., suspensions, syrups, elixirs and the like) can be prepared according to techniques known in the art and can employ the usual media such as water, glycols, oils, alcohols and the like. For example, the dispersion can be simply suspended in an aqueous vehicle, with a typical excipient additive (e.g., 0.5% microcrystalline cellulose) as a suspending agent. Excipients that prevent agglomeration (e.g., poloxamer) also may be added. This type of formulation is especially appropriate for oral dosing in pre-clinical species (e.g., rats). Solid preparations suitable for oral administration (e.g., powders, pills, capsules and tablets) can be prepared according to techniques known in the art and can employ such solid excipients as starches, sugars, kaolin, diluents, lubricants, binders, disintegrating agents and the like. Further description of methods suitable for use in preparing pharmaceutical compositions of the present invention and of ingredients suitable for use in said compositions is provided in Remington's Pharmaceutical Sciences, 20^{th} edition, edited by A. R. Gennaro, Mack Publishing Co., 2000.

### Measurements

The amount of amorphous material in a sample of milled powder can be assessed in a number of ways. Differential Scanning calorimetry (DSC) will show the heat of crystallisation in a sample containing amorphous material. Alternatively the change in weight of a sample exposed to an atmosphere of controlled temperature and humidity can give a measure of the change in amorphous content. In both methods the apparatus is calibrated using samples of known crystalline content and the unknown sample measured by comparing the magnitude of the measurement for the unknown with the known samples.

Surface area can be measured by gas absorption using the Brunauer-Emmet-Teller method or by air permeametry using the Blaine method. Results given here relate to the latter method which is described in the standard method of the l'Association Francaise de Normalisation (AFNOR) no P 15-442 March 1987.

Weight change under controlled relative humidity is measured using a DVS 1 dynamic vapour sorption apparatus. A small weighed sample is placed in a microbalance pan and held at constant temperature of 25° C. and a relative humidity of 75%. Weight change is measured as a function of time over a period of at least 5 hours. The plot of weight v time shows a peak which is proportional to the proportion of amorphous material present. The equipment is calibrated with samples of known amorphous content produced by mixing fully crystalline and fully amorphous materials.

DSC measurements can be carried out using a Seiko RDC 220 system. The sample is weighed into the measuring pan and held at a temperature below the recrystallisation temperature for 30 minutes under a flow of dry nitrogen to remove any surface moisture. The sample was then heated at a constant rate of 20° C. per minute. The exothermic peak due to recrystallisation is measured. As above the method is calibrated using samples of known amorphous content.

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

### EXAMPLES

### Example 1

Indomethacin (Sigma-Aldrich, St. Louis, Mo.), HPMCAS-LF polymer (hydroxypropylmethylcellulose acetate succinate Grade LF; Shin-Etsu Chemical Co., Ltd., Tokyo, Japan) and Neusilin® (Fuji Chemical Industry Co., Ltd., Nakaniikawa-gun, Japan) were weighed into the zirconium milling cells of a Retsch mill (Retsch GmbH, Haan Germany). The ratio for this dispersion is 1:1:1 Indomethacin : Neusilin®: Secondary Polymer. The sample quantity for each milling cell should not exceed more than approximately 20% of the volume of the milling cells depending upon the bulk density of the powders. A zirconium grinding ball (10-12 mm ball for the 10 mL milling cell and 20 mm ball for the 35 mL cell) was placed in each of the milling cell. The milling cells were placed on the Retsch Mill and the mixture was milled at 25-30 Hz for 90 minutes (Note: the milling time can vary from 15-120 minutes; however, most drug samples achieve amorphization within 90 minutes). After 90 minutes, the amorphous solids were removed from the milling cells. Any residual solids were carefully removed using a spatula.

The resulting solids were confirmed to be fully amorphous using instrumental techniques such as X-ray Powder Diffraction, DSC, microscopy, etc. To further confirm amorphization, improved solubility and physical stability of the amorphous phase, a dissolution experiment of the amorphous solids was run in simulated intestinal fluid (fasted state, pH 6.5) to study the solubility/dissolution over the course of four hours. See Dressman et al., 2000, Eur. J. Pharm Sci. 11:73-80. To demonstrate improved solubility in FaSSIF, the solubility results of the amorphous dispersion were compared to the FaSSIF solubility of the crystalline drug over the same time course. Drug recrystallization was also monitored through the above mentioned instrumental techniques.

TABLE 1
(Indomethacin)

| Formulation | Time (hours) | Solubility (mg/mL) |
|---|---|---|
| Indomethacin:Neusilin ® (1:2) | 1 | 2.71 |
| | 2 | 1.05 |
| | 3 | 0.87 |
| | 4 | 0.42 |
| Indomethacin:Neusilin ®:Secondary | 1 | 2.57 |
| Polymer (1:1:1) | 2 | 2.66 |
| | 3 | 2.53 |
| | 4 | 2.73 |

These experiments confirmed that the addition of a secondary polymer to the matrix enabled complete amorphization as opposed to the Indomethacin : Neusilin® formulation which contained seed crystals which subsequently led to a drop in solubility. Further confirmation was seen in the simulated intestinal fluid (fasted state, pH 6.5) solubility data in Table 1, which showed that the solubility remained constant over the time course studied.

### Example 2

Two other compounds (Compound 2 and Compound 3) were tested according to the experimental procedures described in Example 1. Simulated intestinal fluid (fasted state, pH 6.5) solubility data for Compound 3 and Compound 2 is shown in Tables 2 and 3, respectively.

TABLE 2
(Compound 3)

| Formulation | Time (hours) | Solubility (mg/mL) |
|---|---|---|
| Compound 3:Neusilin ® (1:2) | 1 | 0.045 |
| | 2 | 0.042 |
| | 3 | 0.038 |
| | 4 | 0.032 |
| Compound 3:Neusilin ®:Secondary | 1 | 0.12 |
| Polymer (1:1:1) | 2 | 0.22 |
| | 3 | 0.32 |
| | 4 | 0.52 |

TABLE 3
(Compound 2)

| Formulation | Time (hours) | Solubility (mg/mL) |
|---|---|---|
| Compound 2:Neusilin ® (1:2) | 1 | 0.025 |
| | 2 | 0.016 |
| | 3 | 0.019 |
| | 4 | 0.018 |
| Compound 2:Neusilin ®:Secondary | 1 | 0.25 |
| Polymer (1:1:1) | 2 | 0.25 |
| | 3 | 0.23 |
| | 4 | 0.22 |

As shown in Tables 2 and 3, the addition of a secondary polymer not only suppressed crystallization to the crystalline API phase, but also unexpectedly showed better initial solubility than the Neusilin® system alone.

1. A method for producing a substantially amorphous stable drug product comprising preparing an amorphous dispersion of an active pharmaceutical ingredient (API) in the presence of an inorganic matrix and a secondary polymer under conditions such that the final drug product has a crystalline content of less than 5%.

2. The method of claim 1, wherein said preparing is by spray drying, extrusion, or milling.

3. The method of claim 2, wherein said preparing is by milling.

4. The method of claim 1, wherein the inorganic matrix is a silicate, a calcium phosphate, or an inorganic clay.

5. The method of claim 4, wherein the silicate is magnesium aluminosilicate.

6. The method of claim 5, wherein the silicate is magnesium aluminometasilicate.

7. The method of claim 1, wherein the secondary polymer is a cellulose, acrylate, poloxamer, polyvinylpyrollidine, polyethylene glycol, aminosaccharide, or polyethylene oxide.

8. The method of claim 7, wherein the cellulose is ethyl(hydroxyethyl)cellulose, hydroxypropyl methylcellulose, or hydroxyethyl cellulose optionally modified with one or more hydrophobic/hydrophilic groups or a methacrylic acid copolymer.

9. The method of claim 7, wherein the secondary polymer is hydroxypropyl methylcellulose functionalized with a carboxylic acid.

10. The method of claim 9, wherein the secondary polymer is hydroxypropyl methylcellulose acetate succinate or hydroxypropyl methylcellulose phthalate.

11. The method of claim 1, wherein the drug product has a crystalline content of less than 2%.

12. The method of claim 11, wherein the drug product has a crystalline content of less than 1%.

13. The method of claim 1, wherein the drug product is megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, budesnonide, progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, or pemoline.

14. The method of claim 13, wherein the drug product is megestrol acetate, ciprofloxan, itroconazole, lovastatin, simvastatin, omeprazole, phenytoin, ciprofloxacin, cyclosporine, ritonavir, carbamazepine, carvendilol, clarithromycin, diclofenac, etoposide, or budesnonide.

15. The method of claim 1, wherein the drug product is 5″-chloro-N-[(5,6-dimethoxypyridin-2-yl)methyl]-2,2′:5′,3″-terpyridine-3′-carboxamide, N¹-(1-cyanocyclopropyl)-4-fluoro-N²-{(1S)-2,2,2-trifluoro-1-[4′-methylsulfonyl]-1,1′-biphenyl-4-yl}ethyl}-L-leucinamide, or 3-Chloro-5-{[5-chloro-1-(1H-pyrazolo[3,4-b]pyridin-3-ylmethyl)-1H-indazol-4-yl]oxy}benzonitrile.

16. Amorphous drug product produced by the method of claim 1.

17. The amorphous drug product of claim 16 containing less than 1% crystalline content.

18. Amorphous drug product comprising an API, an inorganic matrix and a secondary polymer.

19. The amorphous drug product of claim 18 containing less than 1% crystalline content.

20. A formulation containing the amorphous drug product of claim 16 in the form of a liquid suspension or solid dosage form.
